(19) United States
(12) Patent Application Publication
(10) Pub. No.: US 2026/0363582 A1
(43) Pub. Date: Nov. 19, 2026

(54) SENSOR ASSEMBLY WITH SPECTROPHOTOMETRIC SENSOR PORTION AND ELECTRODE SENSOR PORTION

(71) Applicant: Becton, Dickinson and Company, Franklin Lakes, NJ (US)

(72) Inventor: Matthew P. Dalene, Santa Ana, CA (US)

(21) Appl. No.: 19/142,488

(22) PCT Filed: Dec. 19, 2023
(86) PCT No.: PCT/US2023/084878

§ 371 Date: Jun. 23, 2025

Related U.S. Application Data
(60) Provisional application No. 63/476,757, filed on Dec. 22, 2022.

(51) Int. Cl. A61B 5/1455; A61B 5/00; A61B 5/257; A61B 5/291

(52) U.S. Cl. CPC A61B 5/14552; A61B 5/14553; A61B 5/257; A61B 5/291; A61B 5/68335; A61B 2562/043; A61B 2562/164; A61B 2562/227

(57) ABSTRACT

A physiological sensor assembly is provided that includes a NIRS sensor portion, an electrode sensor portion, and a portion connector. The NIRS sensor portion has a light source, a light detector, and first and second lateral sides. The electrode sensor portion has at least one electrode configured to sense electrical activity of a patient. The electrode is configured to produce signals representative of the electrical activity. The electrode sensor portion has first and second contact surfaces. The electrode sensor portion is configured such that electrical activity of the patient can be sensed at the first or second contact surface. The portion connector is attached to both the NIRS and electrode sensor portions. The electrode sensor portion can be selectively disposed on the first or the second lateral side of the NIRS sensor portion.

### BACKGROUND OF THE INVENTION

### 1. Technical Field

The present disclosure relates to medical devices that include use noninvasive oximetry sensors and electrodes to the sensors themselves.

### 2. Background Information

Near-infrared spectroscopy (NIRS) is an optical spectrophotometric method that can be used to continuously monitor tissue blood parameters such as oxygenation saturation. The NIRS method is based on the principle that light in the near-infrared range (700 nm to 1,000 nm) can pass easily through skin, bone and other tissues and can be detected thereafter. NIRS systems utilize one or more sensors that each include at least one light source and one or more light detectors for detecting reflected or transmitted light. The light signal is created and sensed in cooperation with a NIRS system that includes a processor and an algorithm for processing signals and the data contained therein. NIRS systems can be configured to determine cerebral oxygenation information. Such systems typically include sensors configured to be positioned on a patient's forehead; e.g., one sensor on each side of the patient's forehead to enable monitoring of each brain hemisphere.

Electroencephalography (“EEG”) is a non-invasive method used to sense and record electrical activity of the brain. EEG devices typically use one or more sensors configured to be disposed on a patient's skin surface. Each of these sensors include at least one electrode for sensing the electrical activity. To measure cerebral electrical activity, it is desirable to position an EEG sensor on the patient's forehead and temple region.

In applications such as, but not limited to, estimating depth of anesthesia (DoA) (sometimes referred to as “depth of consciousness”) it is desirable to monitor both tissue oxygen saturation and cerebral electrical activity. In these applications, the need to position both NIRS sensors and EEG sensors on a patient's forehead can create sensor positioning issues.

What is needed is a sensor that can readily enable a caregiver to simultaneously position a NIRS sensor portion and an EEG sensor portion on a patient's forehead in desirable locations, one that is not configured for application on only one side or the other side of a patient's forehead, and one that is not limited to a particular relative positioning between the NIRS portion and the EEG portion of the sensor.

### SUMMARY

According to an aspect of the present disclosure, a physiological sensor assembly is provided that includes a near-infrared spectroscopy (NIRS) sensor portion, an electrode sensor portion, and a portion connector. The NIRS sensor portion has at least one light source, at least one light detector, a lengthwise extending axis, a first lateral side, and a second lateral side opposite the first lateral side. The electrode sensor portion has at least one electrode configured to sense electrical activity of a patient. The electrode is configured to produce signals representative of sensed electrical activity of the patient. The electrode sensor portion has a first contact surface and a second contact surface opposite the first contact surface. The electrode sensor portion is configured such that electrical activity of the patient can be sensed using the at least one electrode at the first contact surface or at the second contact surface. The portion connector is attached to NIRS sensor portion and to the electrode sensor portion. The portion connector is configured so that the electrode sensor portion can be selectively disposed on the first lateral side of the NIRS sensor portion with the first contact surface disposed to engage a skin surface of the patient or on the second lateral side of the NIRS sensor portion with the second contact surface disposed to engage the skin surface of the patient.

In any of the aspects or embodiments described above and herein, the electrode sensor portion may include a main body portion and a discrete body portion, wherein the portion connector is attached to the main body portion, and the main body portion is connected to the discrete body portion by a flexible connector. The at least one electrode includes at least one electrode with the main body portion and at least one electrode with the discrete body portion.

In any of the aspects or embodiments described above and herein, both the main body portion and the discrete body portion may include a first contact surface and a second contact surface, and the main body portion and the discrete body portion may be both configured such that electrical activity of the patient can be sensed at the first contact surface or at the second contact surface.

In any of the aspects or embodiments described above and herein, the main body portion may include a plurality of electrodes.

In any of the aspects or embodiments described above and herein, the at least one electrode with the main body portion may be configured to sense electrical activity of the patient at the first contact surface or at the second contact surface.

In any of the aspects or embodiments described above and herein, the at least one electrode with the discrete body portion may be configured to sense electrical activity of the patient at the first contact surface or at the second contact surface.

In any of the aspects or embodiments described above and herein, the portion connector may include electrical circuitry providing an electrical path through the portion connector for electrical signals from the at least one electrode to pass through the portion connector.

In any of the aspects or embodiments described above and herein, the sensor assembly may include a connector tail attached to the NIRS sensor portion. The connector tail may include electrical circuitry in communication with the electrical circuitry in the portion connector to receive electrical signals from the at least one electrode.

In any of the aspects or embodiments described above and herein, the sensor assembly may include a connector tail attached to the NIRS sensor portion. The connector tail may include electrical circuitry in communication with electrical circuitry in the NIRS sensor portion to receive electrical signals from the at least light detector and/or the at least one light source.

In any of the aspects or embodiments described above and herein, the at least one electrode in the sensor portion may be disposed in a pocket disposed in the electrode sensor portion, and the pocket may be open to the first contact surface and the second contact surface.

In any of the aspects or embodiments described above and herein, the at least one electrode in the sensor portion may include a plurality of electrodes, and a first electrode of the plurality of electrodes may be disposed to sense electrical activity of the patient at the first contact surface, and a second electrode of the plurality of electrodes may be disposed to sense electrical activity of the patient at the second contact surface.

In any of the aspects or embodiments described above and herein, the first electrode of the plurality of electrodes may be disposed in a first pocket disposed in the first contact surface, and a second electrode of the plurality of electrodes may be disposed in a second pocket disposed in the second contact surface.

In any of the aspects or embodiments described above and herein, the at least one electrode may include a plurality of electrodes, and the signals representative of sensed electrical activity of said patient may be in a form acceptable for producing information relating to electroencephalography.

In any of the aspects or embodiments described above and herein, the electrode sensor portion may include a main body portion and a discrete body portion, and the sensor assembly may be configurable in a first configuration wherein the NIRS sensor portion and the main body portion are separated by a first distance, and the sensor assembly may be configurable in a second configuration wherein the NIRS sensor portion and the main body portion are separated by a second distance greater than the first distance.

In any of the aspects or embodiments described above and herein, the electrode sensor portion may include a main body portion and a discrete body portion, and the sensor assembly may be configurable in a first configuration wherein the NIRS sensor portion and the main body portion are longitudinally aligned with one another, and the sensor assembly may be configurable in a second configuration wherein the NIRS sensor portion and the main body portion are longitudinally shifted relative to one another.

In any of the aspects or embodiments described above and herein, the electrode sensor portion may include a main body portion and a discrete body portion and the main body portion may include a first lengthwise extending axis, and the NIRS sensor portion may have a second lengthwise extending axis, and the sensor assembly may be configurable such that the first lengthwise extending axis and the second lengthwise extending axis are non-parallel.

According to another aspect of the present disclosure, a patient monitoring system is provided that includes at least one physiological sensor assembly and a base unit. The sensor assembly includes a NIRS sensor portion, an electrode sensor portion, and a portion connector. The NIRS sensor portion has at least one light source, at least one light detector, a lengthwise extending axis, and first and second lateral sides. The electrode sensor portion has at least one electrode configured to sense electrical activity of a patient. The electrode is configured to produce signals representative of sensed electrical activity of the patient. The electrode sensor portion has a first contact surface and a second contact surface opposite the first contact surface. The electrode sensor portion is configured such that electrical activity of the patient can be sensed using the at least one electrode at the first contact surface or at the second contact surface. The portion connector is attached to NIRS sensor portion and to the electrode sensor portion. The portion connector is configured so that the electrode sensor portion can be selectively disposed on the first lateral side of the NIRS sensor portion with the first contact surface disposed to engage a skin surface of the patient or on the second lateral side of the NIRS sensor portion with the second contact surface disposed to engage the skin surface of the patient. The base unit has a system controller in communication with the at least one sensor assembly. The controller includes at least one processor and a memory device configured to store instructions. The instructions when executed cause the controller to control the NIRS sensor portion of the at least one physiological sensor assembly NIRS sensing device to sense a patient's tissue and produce information relating to an oxygen saturation level of the sensed patient tissue; and determine information relating to electrical activity within the sensed tissue of the patient.

In any of the aspects or embodiments described above and herein, the information relating to electrical activity within the sensed tissue of the patient may be electroencephalography (EEG) information.

In any of the aspects or embodiments described above and herein, the at least one physiological sensor assembly may include a first sensor assembly and a second sensor assembly that are the same. The first sensor assembly may be configured in a first arrangement for application to a left side of a said patient forehead and the second sensor assembly may be configured in a second arrangement for application to a right side of the patient forehead.

In any of the aspects or embodiments described above and herein, in the first arrangement the first sensor assembly may have the electrode sensor portion disposed on the first lateral side of the NIRS sensor portion of the first sensor assembly, and in the second arrangement the second sensor assembly may have the electrode sensor portion disposed on the second lateral side of the NIRS sensor portion of the second sensor assembly.

The foregoing features and elements may be combined in various combinations without exclusivity, unless expressly indicated otherwise. These features and elements as well as the operation thereof will become more apparent in light of the following description and the accompanying drawings. It should be understood, however, the following description and drawings are intended to be exemplary in nature and non-limiting.

### BRIEF DESCRIPTION OF THE DRAWINGS

FIG. 1 is a perspective view of a sensor assembly embodiment.

FIG. 2 is a diagrammatic top view of a sensor assembly embodiment.

FIG. 3 is a diagrammatic end view of a sensor assembly embodiment, illustrating an electrode sensor portion on both lateral sides of a NIRS sensor portion.

FIG. 4 is a diagrammatic top planar view of a NIRS sensor portion embodiment.

FIG. 4A is a side view of the NIRS sensor portion embodiment shown in FIG. 4.

FIG. 5 is a diagrammatic planar view of an electrode sensor portion embodiment.

FIG. 5A is a side view of the electrode sensor portion embodiment shown in FIG. 5.

FIG. 6 is a diagrammatic top view of a sensor assembly embodiment with the electrode sensor portion disposed on a first lateral side of the NIRS sensor portion.

FIG. 6A is a diagrammatic top view of a sensor assembly embodiment shown in FIG. 6, now with the electrode sensor portion disposed on a second lateral side of the NIRS sensor portion.

FIG. 7 is a diagrammatic top view of a sensor assembly embodiment, illustrating the electrode sensor portion and the NIRS sensor portion spaced apart from one another by a distance D1.

FIG. 7A is a diagrammatic top view of the sensor assembly embodiment shown in FIG. 7, illustrating the electrode sensor portion and the NIRS sensor portion spaced apart from one another by a distance D2.

FIG. 8 is a diagrammatic top view of a sensor assembly embodiment, illustrating the electrode sensor portion longitudinally shifted from the NIRS sensor portion in a first direction.

FIG. 8A is a diagrammatic top view of a sensor assembly embodiment shown in FIG. 8, illustrating the electrode sensor portion longitudinally shifted from the NIRS sensor portion in a second direction.

FIG. 9 is a diagrammatic top view of a sensor assembly embodiment, illustrating the electrode sensor portion and the NIRS sensor portion in a first non-parallel orientation.

FIG. 9A is a diagrammatic top view of a sensor assembly embodiment shown in FIG. 9, illustrating the electrode sensor portion and the NIRS sensor portion in a second non-parallel orientation.

FIG. 10 is a diagrammatic view of a patient monitoring system embodiment.

### DETAILED DESCRIPTION

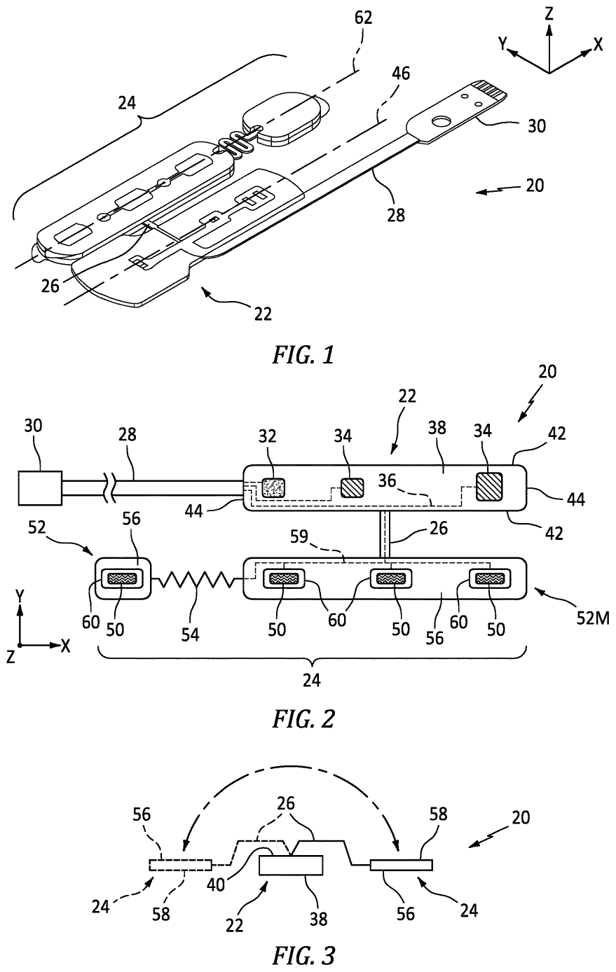

FIG. 1 is a perspective view of a present disclosure sensor assembly 20 embodiment. The sensor assembly 20 includes a spectrophotometric sensor portion (referred to herein after as a NIRS sensor portion 22) and an electrode sensor portion 24, both portions 22, 24 attachable to a patient tissue surface for sensing physiological parameters. The NIRS sensor portion 22 and the electrode sensor portion 24 are connected to one another by a portion connector 26. The sensor assembly 20 includes a connector tail 28 that extends outwardly and terminates in a connector 30. The connector tail 28 is in signal communication with the NIRS sensor portion 22 and the electrode sensor portion 24.

Referring to FIGS. 2-4A, the NIRS sensor portion 22 includes one or more light sources 32, one or more light detectors 34, electrical circuitry 36 that provides signal/electrical communication between the light sources 32 and the connector tail 28, and signal/electrical communication between the light detectors 34 and the connector tail 28. The NIRS sensor portion 22 is flexible enough to conform with a variety of different patient skin surface geometries. The NIRS sensor portion 22 has a contact surface 38 that is configured for contact with the patient's skin and a back surface 40 that is opposite the contact surface 38. In some embodiments, a NIRS sensor portion 22 has lateral side surfaces 42 and end surfaces 44 that extend (along a Z-axis) between the contact surface 38 and the back surface 40. The NIRS sensor portion 22 may be described as having a body defined at least in part by the contact surface, the back surface, the lateral side surfaces, and the end surfaces. The NIRS sensor portion 22 has a lengthwise extending longitudinal axis 46 (e.g., extending along the X-axis) and a widthwise extending axis (e.g., extending along the Y-axis). The light source 32 and light detectors 34 are spaced apart from one another along the lengthwise axis 46.

The light source(s) 32 may include one or more light emitting components (e.g., light emitting diodes (LEDs), laser diodes, etc.) that are selectively operable to produce infrared light at one or more predetermined wavelengths (i.e., light in the range of about seven hundred nanometers (700 nm) to about one thousand nanometers (1,000 nm)). In some embodiments, a light source 32 may also be configured to produce visible light (i.e., light in the wavelength range of about three hundred ninety nanometers (390 nm) to about seven hundred fifty nanometers (750 nm)). The light source 32 is in electrical/signal communication with the connector tail 28 and connector 30. A flexible electrical circuit (“flex circuit”) is a nonlimiting example of electrical circuitry 36 for providing the aforesaid electrical communication. In some embodiments the flex circuit may extend through the connector tail 28 and be in electrical communication with the connector 30.

The light detectors 34 include one or more light responsive transducers (e.g., photodiodes, charge-coupled devices etc.) that are operable to detect light emitted by the light source 32 after such light passes through a portion of the patient's body. The light detectors 34 are in electrical/signal communication with the connector tail 28 and connector 30. In those embodiments wherein the NIRS sensor portion 22 includes more than one light detector 34, the light detector 34 closest to the light source 32 may be referred to hereinafter as the “near detector”, and the light detector 34 farthest from the light source 32 may be referred to hereinafter as the “far detector”. The relative positioning of the light source 32 and the light detectors 34 on the NIRS sensor portion 22 can be varied for different applications; e.g., to distinguish scalp tissue and skull tissue from brain tissue, etc. The NIRS sensor portion 22 is not limited to any particular relative positioning of the light source 32 and the light detectors 34. U.S. Pat. No. 10,321,862, which is hereby incorporated by reference in its entirety, discloses several examples of acceptable light source 32/light detector 34 relative positioning. The relative positioning of the light source 32 and the light detectors 34 may, for example, be selected so that: (1) the light source 32 and the light detectors 34 are substantially linearly aligned along the lengthwise-extending axis; and (2) the separation distances between the light source 32 and each of the light detectors 34 are not the same.

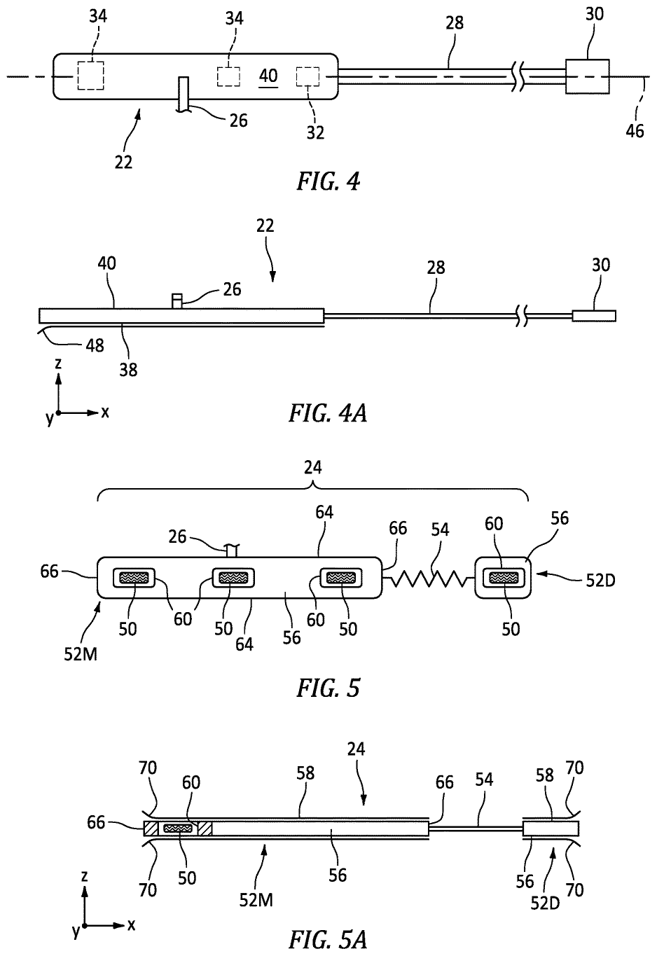

The NIRS sensor portion 22 may include components in addition to the light source(s) 32, light detector(s) 34, and the electrical/signal circuitry 36 (e.g., flex circuit). For example, a NIRS sensor portion 22 may include one or more electrical insulating layers, one of more EMI shielding layers, a support layer, a pad, a cover, a connector tail cover, and the like, and any combination thereof. In some embodiments, an adhesive layer may be disposed on the contact surface to facilitate the NIRS sensor portion 22 being attached to the patient's skin surface. In those embodiments wherein the NIRS sensor portion 22 includes an adhesive layer, the NIRS sensor portion 22 may include a removable protective layer 48 (e.g., see FIG. 4A) disposed on the exposed surface of the adhesive layer to protect it before the NIRS sensor portion 22 is applied.

The present disclosure is not limited to any particular NIRS sensor portion 22 configuration. U.S. Pat. No. 9,888,873 entitled “NIRS Sensor Assembly Including EMI Shielding”, which is hereby incorporated by reference in its entirety, is an example of a NIRS sensor that may be modified for use as a NIRS sensor portion 22 as described herein.

The electrode sensor portion 24 includes one or more electrodes 50 for sensing the electrical activity of a patient. The present disclosure is described herein in terms of an electrode sensor portion 24 configured to function as an electroencephalograph (EEG) sensor. The present disclosure is not, however, limited to an electrode sensor portion 24 configured for EEG sensing purposes.

In the embodiment shown in FIGURES, the electrode sensor portion 24 includes a main body portion 52M and a discrete body portion 52D connected to one another by a flexible connector 54 (e.g., a serpentine arrangement) that permits the variable positioning between the main body portion 52M and the discrete body portion 52D to suit the application; e.g., the main body portion 52M may be applied to the patient's forehead and the discrete body portion 52D may be applied to the patient's temple region. The flexible connector 54 is configured to provide both physical attachment and electrical communication between the main body portion 52M and the discrete body portion 52D. The present disclosure is not limited to an electrode sensor portion 24 having a main body portion 52M and a discrete body portion 52D; e.g., in some embodiments the electrode sensor portion 24 may comprise a single body portion or more than two body portions. The main body portion 52M is configured to have sufficient flexibility to permit it to conform with a variety of different skin surface geometries.

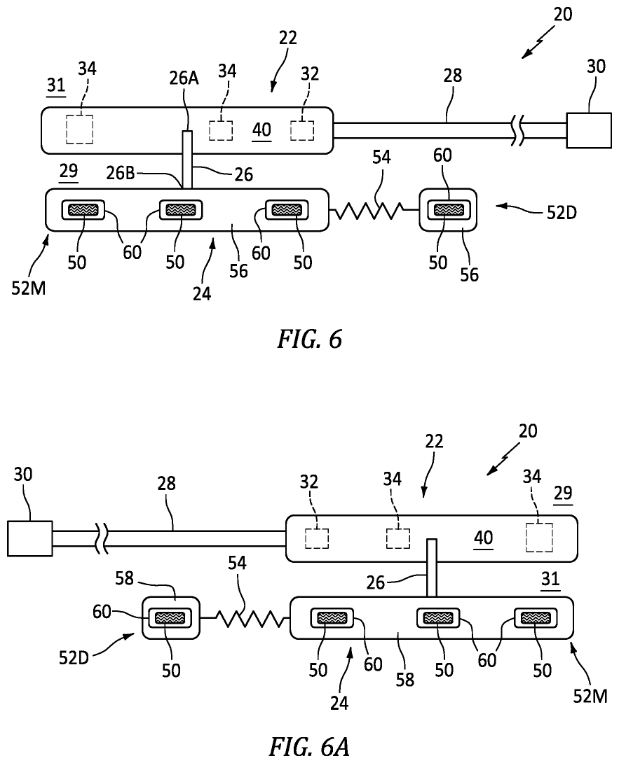

Both the main body portion 52M and the discrete body portion 52D include a first contact surface 56 and a second contact surface 58. As will be detailed herein, the present disclosure sensor assembly 20 can be selectively arranged with the electrode sensor portion 24 disposed on a first lateral side of the NIRS sensor portion 22 (e.g., see FIGS. 3 and 6) or on an opposite second lateral side of the NIRS sensor portion 22 (e.g., see FIG. 6A and FIG. 3 in dotted line). In the first arrangement, the first contact surface 56 is disposed to contact with the patient's skin surface and conversely in the second configuration, the second contact surface 58 is disposed to contact with the patient's skin surface.

In the embodiment shown in FIGURES, the main body portion 52M is shown including three electrodes 50 and the discrete body portion 52D including a single electrode 50. The present disclosure is not limited to this electrode 50 configuration; e.g., the main body portion 52M may include fewer than three electrodes 50 or more than three electrodes 50 and the discrete body portion 52D may include more than one electrode 50.

The main body portion 52M and the discrete body portion 52D of the electrode sensor portion 24 may each be described as being “dual-sided”, meaning that the electrode sensor portion 24 can sense patient electrical activity with its first contact surface 56 in contact with the patient's skin surface or with its second contact surface 58 in contact with the patient's skin surface. Each electrode 50 has at least one sensing surface. In some embodiments, the electrode sensor portion 24 include electrodes 50 capable of sensing patient electrical activity at both the first contact surface 56 or at the second contact surface 58; e.g., which ever contact surface 56, 58 is attached to a patient's skin surface. Alternatively, the main and the discrete body portions 52M, 52D of the electrode sensor portion 24 may include mirror arrangements of electrodes 50, wherein “N” number of electrodes 50 (where “N” is an integer) are disposed at the first contact surface 56 and “N” number of electrodes 50 are disposed at the second contact surface 58; e.g., back to back. The present disclosure is not limited to any electrode 50 arrangement provided the electrode 50 arrangement is capable of sensing patient electrical activity at the first contact surface 56 and at the second contact surface 58.

Each electrode 50 is disposed within a respective pocket 60 formed in the main body portion 52M and the discrete body portion 52D. The pockets 60 are open to the respective contact surface 56, 58. In those embodiments wherein a single electrode 50 is configured to sense patient electrical activity at the first contact surface 56 or the second contact surface 58, the pocket 60 for that electrode 50 is open to both the first contact surface 56 and at the second contact surface 58. In those embodiments wherein the electrode sensor portion 24 includes mirror arrangements of electrodes 50, the respective body portion includes “N” number of pockets 60 disposed in the first contact surface 56 and the same in the opposite second contact surface 58. Typically, a pocket 60 is configured so that the sensing surface of the electrode 50 is disposed below the contact surface 56, 58, leaving room for electrolytic gel between the electrode sensing surface and the contact surface 56, 58 of the body portion 52M, 52D.

In the embodiment shown in FIGURES, the electrodes 50 in the main body portion 52M are aligned along a lengthwise-extending longitudinal axis 62 (e.g., see FIG. 1) extending between the first and second end surfaces. In alternative embodiments, electrodes 50 may not be aligned on a common axis. Electrode sensor portion 24 embodiments may be configured so an electrode 50 operates as a reference electrode, and other electrodes 50 each operate as an active channel electrode. The present disclosure is not limited to any particular operational electrode 50 configuration.

The main body portion 52M example shown in FIGURES is generally rectangularly shaped with the first and second contact surfaces 56, 58 opposite one another. The present disclosure is not limited to a rectangular-shaped main body portion 52M. The main body portion 52M example shown in the FIGURES includes lateral surfaces 64 and end surfaces 66 (e.g., see FIG. 5). The lateral surfaces 64 (e.g., extending along an X-axis) are on opposite lateral sides of the main body portion 52M, and the end surfaces 66 (e.g., extending along a Y-axis) are on opposite ends of the main body portion 52M. In the example embodiment shown in the FIGURES, the lateral surfaces 64 and the end surfaces 66 extend (e.g., in a Z-axis direction) between the first and second contact surfaces 56, 58. The main body portion 52M may be described as having a longitudinal axis that extends between the end surfaces 66, a widthwise axis that extends between the lateral surfaces 64, and a height (or “thickness”) that extends between the first and second contact surfaces 56, 58.

The discrete body portion 52D example shown in the FIGURES is generally rectangularly shaped with first and second contact surfaces 56, 58 opposite one another. The present disclosure is not limited to a discrete body portion 52D that is generally rectangularly shaped. The discrete body portion 52D example includes side surfaces that extend between the first and second contact surfaces 56, 58.

The present disclosure is not limited to the main body portion 52M and discrete body portion 52D configurations described above.

The main body portion 52M and discrete body portion 52D (or the sensor body as it may be otherwise configured) may be configured in a variety of ways. For example, the aforesaid body portions 52M, 52D may be a unitary body formed from a single material, or may be formed from a plurality of layers that collectively form the respective body. In those embodiments that include a unitary body, the body may be configured to contain electrical circuitry 59 (e.g., see FIG. 2), including for example communication lines (e.g., a copper or gold wires) extending from the respective electrodes 50, or a flexible circuit/printed circuit board (PCB) in communication with the electrodes 50 (detailed below). In those embodiments that include a body portion that includes a plurality of layers, the electrical communication lines or flexible circuit/PCB may form, or be disposed in a layer, or be disposed between layers of the body portion. In some embodiments, the main body portion 52M may be configured differently from the discrete body portion 52D (e.g., different materials, layers, and the like) or both portions 52M, 52D may have the same configuration.

The body portions 52M, 52D may comprise one or more generally flexible materials (e.g., polymeric materials such as a polyimide). The degree to which a body portion is flexible can vary depending on the intended application of the sensor assembly 20; e.g., sufficiently flexible to readily correspond to curvature of the application site, etc. In some embodiments, a body portion 52M, 52D may comprise a material that is breathable (air and/or moisture) to increase comfort and to facilitate retention of the sensor body on the patient's skin.

In some embodiments, the body portions 52M, 52D may each include a removable protection layer 70 (e.g., see FIG. 5A) that is initially disposed in contact with the respective contact surface 56, 58 but is intended is to be removed prior to use. The removable protection layer 70 may be adhered to the respective contact surface using an adhesive.

In some embodiments, the body portions 52M, 52D may be configured for attachment to the patient's skin surface via a layer of adhesive disposed on a respective contact surface 56, 58. The present disclosure is not limited to using adhesive for maintaining contact between the respective body portion 52M, 52D and the patient's skin surface. In alternative embodiments, the respective body portions 52M, 52D may be configured to maintain contact with the patient's skin surface via an independent element such as an elastic member, a bandage member, a strap, a cap, any combination of the same, or other devices for fastening sensors to a patient's body or skin known in the art. The present disclosure is not limited to any particular configuration for maintaining contact between the respective sensor body portion 52M, 52D and the patient's skin surface.

The present disclosure sensor assemblies include an electrolytic gel disposed in communication with the sensing surface of each electrode 50. The electrolytic gel is configured to facilitate electrical signal transmission from patient to the electrode 50; e.g., to improve electrical signal conduction between the patient and the sensing surface of the electrode 50. In some embodiments, the present disclosure may use a material that may be more appropriately characterized as a fluid rather than a gel. To facilitate the description herein, the term “electrolytic gel” is intended to include electrolytic materials that are known and may be used in with the present disclosure (e.g., to provide an electrical communication path between sensing surface of each electrode 50 and a patient's skin surface), including those electrolytic materials in gel form or fluid form. In some embodiments a sensor assembly 20 may include a porous medium such as an open cell foam (e.g., a reticulated foam) disposed within a pocket 60. The porous medium may be disposed in the region of the pocket 60 between the electrode sensing surface and the plane across the opening of the pocket 60 that is coplanar with the contact surface 56, 58. The porous medium may facilitate retention of the electrolytic gel within the pocket 60.

As indicated above, the present disclosure sensor assembly 20 includes a portion connector 26 that connects the NIRS sensor portion 22 and the electrode sensor portion 24. The portion connection 26 is flexible. The portion connector 26 includes a first end 26A (e.g., see FIG. 6) that is in communication with the NIRS sensor portion 22 (e.g., at the back surface 40) and a second end 26B that is in communication with the electrode sensor portion 24 (e.g., at a side surface). In the embodiments shown in the FIGURES, the portion connector 26 extends generally from the middle of the NIRS sensor portion 22 to the middle of the electrode sensor portion 24. The portion connector 26 is configured to physically connect the NIRS sensor portion 22 and the electrode sensor portion 24 and to provide a signal communication path from the electrode sensor portion 24 to the NIRS sensor portion 22. As will be detailed below, the portion connector 26 may also be configured to permit positional degrees of freedom between the NIRS sensor portion 22 and the electrode sensor portion 24. Within the NIRS sensor portion 22, signal communications from the electrode sensor portion 24 may be routed through a portion of a flex circuit within the NIRS sensor portion 22 to the connector tail 28, or may be routed through one or more electrical communication lines (e.g., a copper or gold wires) to the connector tail 28.

The portion connector 26 is configured to permit the attached the electrode sensor portion 24 to be disposed on the first lateral side 29 of the NIRS sensor portion 22 (e.g., see FIG. 6) or on the opposite second lateral side 31 of the NIRS sensor portion 22 (e.g., see FIG. 6A); e.g., by rotating the electrode sensor portion 24 from one lateral side to the other.

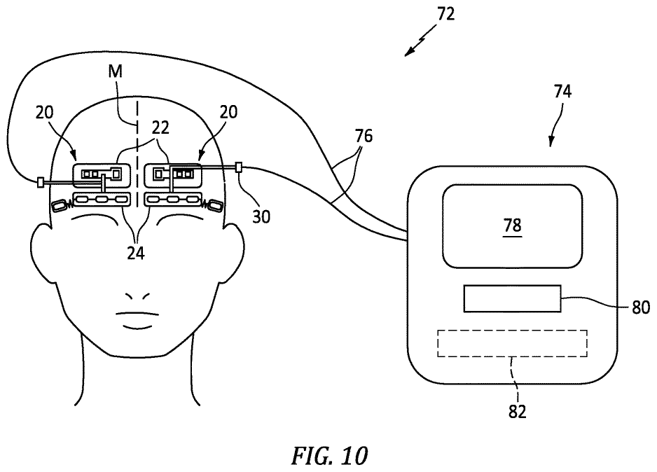

In applications where the electrode sensor portion 24 is configured to operate as an EEG sensor, it is desirable to apply the main body portion 52M of the electrode sensor portion 24 above the patient's eyebrow on one side of the patient's forehead and apply the discrete body portion 52D in the temple region of the patient on the same size of the patient's head. In this manner, electrical activity associated with one hemisphere of the patient's brain can be sensed. At the same time, it is desirable to place the NIRS sensor portion 22 on the same side of the patient forehead, above the electrode sensor portion 24. The dual-sided nature of the present disclosure electrode sensor portion 24 enables the same sensor assembly 20 to be used on both the right side of the patient's forehead and on the left side of the patient's forehead by rotating the electrode sensor portion 24 from one lateral side of the NIRS sensor portion 22 to the other side. FIG. 10 illustrates two of the same present disclosure sensor assemblies mounted on a patient's forehead. The sensor assembly 20 disposed on the left side of the patient's forehead has the electrode sensor portion 24 disposed on one lateral side of the NIRS sensor portion 22 and the sensor assembly 20 disposed on the right side of the patient's forehead has the electrode sensor portion 24 disposed on the opposite lateral side of the NIRS sensor portion 22.

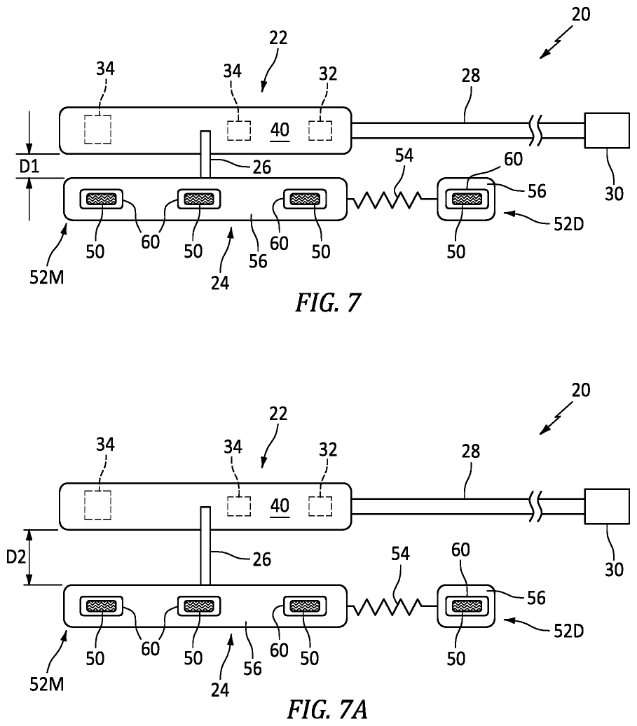

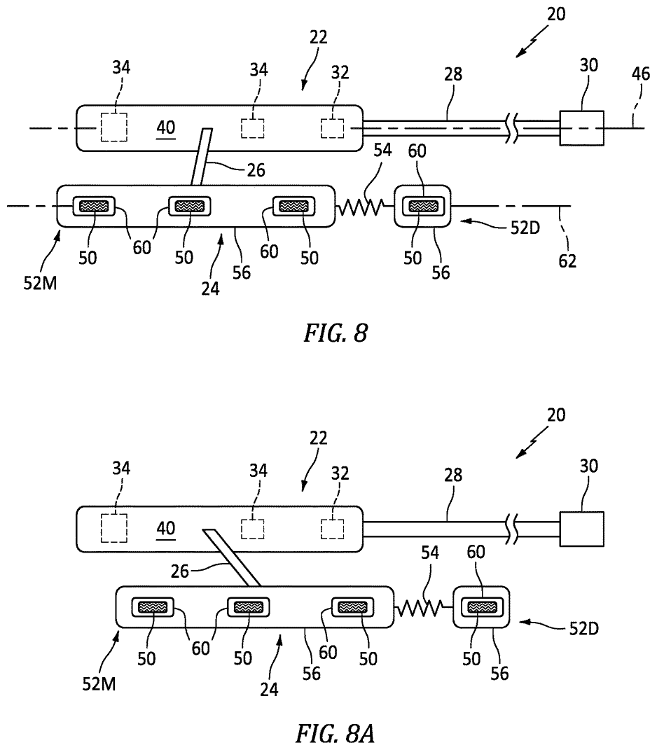

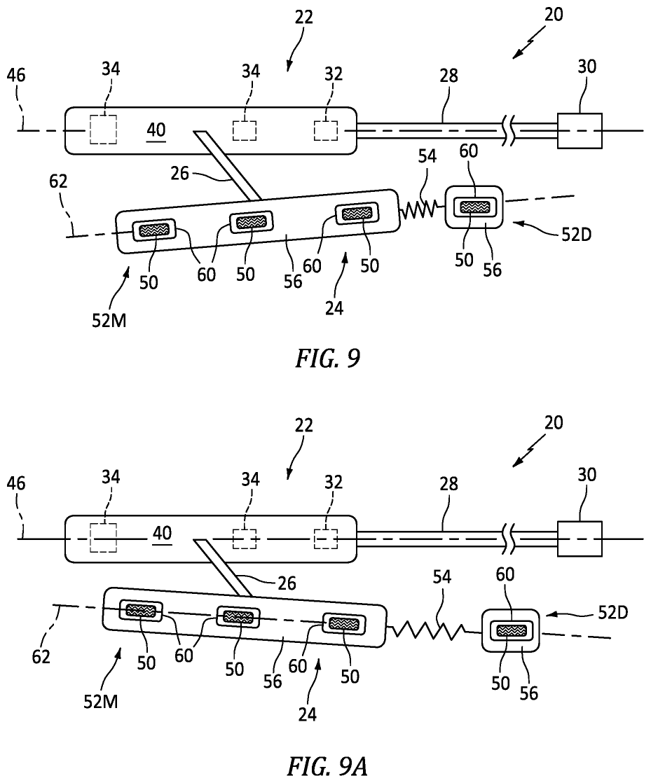

The present disclosure sensor assembly 20 provides additional utility beyond a single sensor assembly 20 that can be used on either side of a patient's forehead. The portion connector 26 disposed between the NIRS sensor portion 22 and the electrode sensor portion 24 also permits positional degrees of freedom between the NIRS sensor portion 22 and the electrode sensor portion 24. These degrees of freedom permit the user to selectively place each of the NIRS sensor portion 22 and the electrode sensor portion 24 on the patient's forehead. A person of skill in the art will recognize that patient “features” (e.g., hairlines, forehead contour, skin bruising, and the like) will often influence where a user will wish to apply a NIRS sensor portion 22 and/or an electrode sensor portion 24. The present disclosure sensor assembly 20 permits the user to place the NIRS sensor portion 22 and/or an electrode sensor portion 24 in desirable positions and to avoid problematic positions. As a first example, FIG. 7 diagrammatically illustrates a sensor assembly 20 wherein the NIRS sensor portion 22 and the electrode sensor portion 24 are separated by a distance “D1”. FIG. 7A diagrammatically illustrates the same sensor assembly 20, and now illustrates the NIRS sensor portion 22 and the electrode sensor portion 24 separated by a distance “D2”, where D2 is greater than D1 (D2>D1). Hence, the separation distance between the NIRS sensor portion 22 and the electrode sensor portion 24 can be selectively chosen by the user based on the application. As another example, FIG. 8 diagrammatically illustrates a sensor assembly 20 wherein the NIRS sensor portion 22 is longitudinally shifted relative to the electrode sensor portion 24 in a first direction (e.g., the center point of the NIRS sensor portion 22 is longitudinally shifted relative to the CenterPoint of the main body portion 52M of the electrode sensor portion 24), and FIG. 8A diagrammatically illustrates the same sensor assembly 20 with the NIRS sensor portion 22 longitudinally shifted relative to the electrode sensor portion 24 in a second direction opposite the first direction. Hence, the specific relative longitudinal positions of the NIRS sensor portion 22 and the electrode sensor portion 24 can be selectively chosen by the user based on the application. As yet another example, the NIRS sensor portion 22 and the electrode sensor portion 24 in the sensor assemblies shown in FIGS. 7, 7A, 8, and 8A have longitudinal axes 46, 62 that are substantially parallel (e.g., see FIG. 8). FIGS. 9 and 9A diagrammatically illustrate sensor assemblies wherein the longitudinal axes 46, 62 of the NIRS sensor portion 22 and the electrode sensor portion 24 are non-parallel; e.g., in FIG. 9, the axes 46, 62 are skewed in a first direction and in FIG. 9A the longitudinal axes 46, 62 are skewed in an opposite second direction. Hence, the longitudinal orientation of the NIRS sensor portion 22 relative to the electrode sensor portion 24 (and vice versa) can be selectively chosen by the user based on the application. The examples diagrammatically shown in FIGS. 7-9A are provided to illustrate the positional freedom provided to the user by the present disclosure sensor assembly 20. The present disclosure sensor assemblies can be positioned with any combination of these relative positions.

FIG. 10 diagrammatically illustrates an exemplary patient monitoring system 72. The system 72 includes a base unit 74, at least one sensor assembly 20, and one or more communication lines 76 that communicatively connect the sensor assembly(ies) 20 with the base unit 74; e.g., connect to the connector 30 disposed at the terminal end of the respective connector tail 28. The communication lines 76 may include electrical conductors configured to conduct electrical signals between sensor assemblies 20 and the base unit 74. In some embodiments, additional hardware including signal amplification devices may be in communication with the communications lines. The base unit 74 may include a display device 78, an input device 80, and a system controller 82. Examples of acceptable display devices 78 include LED screens, LCD screens, and the like. Examples of acceptable input devices 80 include a keyboard, a touch screen, a voice commanded unit, or the like.

The system controller 82 is in communication with other system components including the sensor assemblies 20 and the like. The system controller 82 may be in communication with system components to control the operation of the respective component and/or to receive signals from and/or transmit signals to that component to perform the functions described herein. The system controller 82 may include any type of computing device, computational circuit, processor(s), CPU, computer, or the like capable of executing a series of instructions that are stored in memory. The instructions may include an operating system, and/or executable software modules such as program files, system data, buffers, drivers, utilities, and the like. The executable instructions may apply to any functionality described herein to enable the system to accomplish the same algorithmically and/or coordination of system components. The system controller 82 includes or is in communication with one or more memory devices. The present disclosure is not limited to any particular type of memory device, and the memory device may store instructions and/or data in a non-transitory manner. Examples of memory devices that may be used include read-only memory, random access memory, volatile memory, non-volatile memory, static memory, dynamic memory, flash memory, cache memory, and/or any device that stores digital information. The system controller 82 may include, or may be in communication with, an input device that enables a user to enter data and/or instructions, and may include, or be in communication with, an output device configured, for example to display information (e.g., a visual display or a printer), or to transfer data, etc. The example system shown in FIG. 2 has communication lines that connect the sensor assembly(ies) 20 with the base unit. In alternative embodiments, the sensor assemblies 20 and base unit/system controller 82 may be configured to communicate via a wireless connection.

During use of the present disclosure system and/or sensor assemblies, a user (e.g., physician, nurse, etc.) may remove a sensor assembly 20 from its packaging and determine which side of the patient's forehead the user wishes to apply the sensor assembly 20. Based on that determination, the user can then arrange the electrode sensor portion 24 relative to the NIRS sensor portion 22 (e.g., rotate the electrode sensor portion 24 from the first lateral side of the NIRS sensor portion 22 to the second lateral side if need be). Once the correct arrangement is determined, the user may then remove the protective layer 70 from the first or second contact surfaces 56, 58 of the main and discrete body portions 52M, 52D (whichever is to be applied to the patient's skin surface) and apply the main body portion 52M to the patient's forehead above the eyebrow and the discrete body portion 52D to the patient's temple region on that side of the forehead. The user may then remove the protective layer 48 from the NIRS sensor portion 22 and apply the NIRS sensor portion 22 to the patient's forehead above the electrode sensor portion 24. As indicated above, the present disclosure sensor assembly 20 permits the electrode sensor portion 24 and the NIRS sensor portion 22 to be selectively positioned relative to one another in desirable positions and to avoid problematic positions. The same process can be followed for a second present disclosure sensor assembly 20 on the opposite side of the patient's forehead. In this manner, the pair of sensor assemblies 20 can be used to collect data from each hemisphere of the patient's brain. The sensor assemblies 20 can be connected to the base unit 74 of the patient monitoring system 72 by connecting the communication lines 76 from the base unit 74 to the connector 30 of each respective sensor assembly 20. During operation of the patient monitoring system 72, stored instructions accessible by the system controller 82 can be used to control the operation of the NIRS sensor portion light source(s) 32 and can be used to receive and process signal communications from the NIRS sensor portion 22 and electrode sensor portion 24 of each sensor assembly 20. The signals representative of the electrical activity sensed from the patient can be processed according to known algorithms for producing EEG data. The signals representative of light detected by the light detectors 34 of the NIRS sensor portions 22 (i.e., light from the respective light source 32 that has passed through patient tissue) can be processed according to known algorithms and methodologies for producing tissue oxygen saturation data and the like. Nonlimiting examples of algorithms and methodologies for producing tissue oxygen saturation data and the like are disclosed in U.S. Pats. No. 7,072,701; 8,078,250; 8,428,674; 8,788,004; 8,897,848; 8,923,943; 8,965,472; 9,456,773; 9,848,808; 10,117,610 10,261,010; 10,485,462; 10,918,321; and 11,454,589, each of which is hereby incorporated by reference in its entirety. These issued patents, commonly assigned with the present application, represent non-limiting examples of algorithms and methods that may be utilized with the present disclosure.

While the principles of the disclosure have been described above in connection with specific apparatuses and methods, it is to be clearly understood that this description is made only by way of example and not as limitation on the scope of the disclosure. Specific details are given in the above description to provide a thorough understanding of the embodiments. However, it is understood that the embodiments may be practiced without these specific details.

The singular forms “a,” “an,” and “the” refer to one or more than one, unless the context clearly dictates otherwise. For example, the term “comprising a sample” includes single or plural samples and is considered equivalent to the phrase “comprising at least one sample.” The term “or” refers to a single element of stated alternative elements or a combination of two or more elements unless the context clearly indicates otherwise. As used herein, “comprises” means “includes.” Thus, “comprising A or B,” means “including A or B, or A and B,” without excluding additional elements.

It is noted that various connections are set forth between elements in the present description and drawings (the contents of which are included in this disclosure by way of reference). It is noted that these connections are general and, unless specified otherwise, may be direct or indirect and that this specification is not intended to be limiting in this respect. Any reference to attached, fixed, connected or the like may include permanent, removable, temporary, partial, full and/or any other possible attachment option.

No element, component, or method step in the present disclosure is intended to be dedicated to the public regardless of whether the element, component, or method step is explicitly recited in the claims. No claim element herein is to be construed under the provisions of 35 U.S.C. 112(f) unless the element is expressly recited using the phrase “means for.” As used herein, the terms “comprise”, “comprising”, or any other variation thereof, are intended to cover a non-exclusive inclusion, such that a process, method, article, or apparatus that comprises a list of elements does not include only those elements but may include other elements not expressly listed or inherent to such process, method, article, or apparatus.

While various inventive aspects, concepts and features of the disclosures may be described and illustrated herein as embodied in combination in the exemplary embodiments, these various aspects, concepts, and features may be used in many alternative embodiments, either individually or in various combinations and sub-combinations thereof. Unless expressly excluded herein all such combinations and sub-combinations are intended to be within the scope of the present application. Still further, while various alternative embodiments as to the various aspects, concepts, and features of the disclosures-such as alternative materials, structures, configurations, methods, devices, and components, and so on-may be described herein, such descriptions are not intended to be a complete or exhaustive list of available alternative embodiments, whether presently known or later developed. Those skilled in the art may readily adopt one or more of the inventive aspects, concepts, or features into additional embodiments and uses within the scope of the present application even if such embodiments are not expressly disclosed herein. For example, in the exemplary embodiments described above within the Detailed Description portion of the present specification, elements may be described as individual units and shown as independent of one another to facilitate the description. In alternative embodiments, such elements may be configured as combined elements. It is further noted that various method or process steps for embodiments of the present disclosure are described herein. The description may present method and/or process steps as a particular sequence. However, to the extent that the method or process does not rely on the particular order of steps set forth herein, the method or process should not be limited to the particular sequence of steps described. As one of ordinary skill in the art would appreciate, other sequences of steps may be possible. Therefore, the particular order of the steps set forth in the description should not be construed as a limitation.

Additionally, even though some features, concepts, or aspects of the disclosures may be described herein as being a preferred arrangement or method, such description is not intended to suggest that such feature is required or necessary unless expressly so stated. Still further, exemplary or representative values and ranges may be included to assist in understanding the present application, however, such values and ranges are not to be construed in a limiting sense and are intended to be critical values or ranges only if so expressly stated.

The treatment techniques, methods, and steps described or suggested herein or in references incorporated herein may be performed on a living animal or on a non-living simulation, such as on a cadaver, cadaver heart, anthropomorphic ghost, or simulator (e.g., with the body parts, or tissue being simulated).

Any of the various systems, devices, apparatuses, etc. in this disclosure may be sterilized (e.g., with heat, radiation, ethylene oxide, hydrogen peroxide) to ensure they are safe for use with patients, and the methods herein may comprise sterilization of the associated system, device, apparatus, etc. ; e.g., with heat, radiation, ethylene oxide, hydrogen peroxide.

1. A physiological sensor assembly, comprising:
a near-infrared spectroscopy (NIRS) sensor portion having at least one light source, at least one light detector, a lengthwise extending axis, a first lateral side, and a second lateral side opposite the first lateral side; and
an electrode sensor portion having at least one electrode configured to sense electrical activity of a patient, the electrode configured to produce signals representative of sensed electrical activity of said patient, the electrode sensor portion having a first contact surface and a second contact surface opposite the first contact surface, wherein the electrode sensor portion is configured such that said electrical activity of said patient can be sensed using the at least one electrode at the first contact surface or at the second contact surface; and
a portion connector attached to NIRS sensor portion and to the electrode sensor portion, wherein the portion connector is configured so that the electrode sensor portion can be selectively disposed on the first lateral side of the NIRS sensor portion with the first contact surface disposed to engage a skin surface of the patient or on the second lateral side of the NIRS sensor portion with the second contact surface disposed to engage the skin surface of the patient.

2. The sensor assembly of claim 1, wherein the electrode sensor portion includes a main body portion and a discrete body portion, wherein the portion connector is attached to the main body portion, the main body portion is connected to the discrete body portion by a flexible connector, and the at least one electrode includes at least one said electrode with the main body portion and at least one said electrode with the discrete body portion.

3. The sensor assembly of claim 2, wherein both the main body portion and the discrete body portion include a said first contact surface and a said second contact surface, and the main body portion and the discrete body portion are both configured such that said electrical activity of said patient can be sensed at the first contact surface or at the second contact surface.

4. The sensor assembly of claim 2, wherein the main body portion includes a plurality of said electrodes.

5. The sensor assembly of claim 2, wherein the at least one said electrode with the main body portion is configured to sense said electrical activity of said patient at the first contact surface or at the second contact surface.

6. The sensor assembly of claim 2, wherein the at least one said electrode with the discrete body portion is configured to sense said electrical activity of said patient at the first contact surface or at the second contact surface.

7. The sensor assembly of claim 1, wherein the portion connector includes electrical circuitry providing an electrical path through the portion connector for electrical signals from the at least one electrode to pass through the portion connector.

8. The sensor assembly of claim 7, further comprising a connector tail attached to the NIRS sensor portion, the connector tail including electrical circuitry in communication with the electrical circuitry in the portion connector to receive said electrical signals from the at least one electrode.

9. The sensor assembly of claim 7, further comprising a connector tail attached to the NIRS sensor portion, the connector tail including electrical circuitry in communication with electrical circuitry in the NIRS sensor portion to receive said electrical signals from the at least light detector, or the at least one light source, or both.

10. The sensor assembly of claim 1, wherein the at least one electrode in the sensor portion is disposed in a pocket disposed in the electrode sensor portion, the pocket open to the first contact surface and the second contact surface.

11. The sensor assembly of claim 1, wherein the at least one electrode in the sensor portion includes a plurality of electrodes, and a first electrode of the plurality of electrodes is disposed to sense said electrical activity of said patient at the first contact surface, and a second electrode of the plurality of electrodes is disposed to sense said electrical activity of said patient at the second contact surface.

12. The sensor assembly of claim 11, wherein the first electrode of the plurality of electrodes is disposed in a first pocket disposed in the first contact surface, and a second electrode of the plurality of electrodes is disposed in a second pocket disposed in the second contact surface.

13. The sensor assembly of claim 1, wherein the at least one electrode includes a plurality of electrodes, and the signals representative of sensed electrical activity of said patient are in a form acceptable for producing information relating to electroencephalography.

14. The sensor assembly of claim 1, wherein the electrode sensor portion includes a main body portion and a discrete body portion; and
wherein the sensor assembly is configurable in a first configuration wherein the NIRS sensor portion and the main body portion are separated by a first distance; and
wherein the sensor assembly is configurable in a second configuration wherein the NIRS sensor portion and the main body portion are separated by a second distance greater than the first distance.

15. The sensor assembly of claim 1, wherein the electrode sensor portion includes a main body portion and a discrete body portion; and
wherein the sensor assembly is configurable in a first configuration wherein the NIRS sensor portion and the main body portion are longitudinally aligned with one another; and
wherein the sensor assembly is configurable in a second configuration wherein the NIRS sensor portion and the main body portion are longitudinally shifted relative to one another.

16. The sensor assembly of claim 1, wherein the electrode sensor portion includes a main body portion and a discrete body portion and the main body portion includes a first lengthwise extending axis; and
wherein the NIRS sensor portion has a second lengthwise extending axis; and
wherein the sensor assembly is configurable such that the first lengthwise extending axis and the second lengthwise extending axis are non-parallel.

17. A patient monitoring system, comprising:
at least one physiological sensor assembly, comprising:
a near-infrared spectroscopy (NIRS) sensor portion having at least one light source, at least one light detector, a lengthwise extending axis, a first lateral side, and a second lateral side opposite the first lateral side; and
an electrode sensor portion having at least one electrode configured to sense electrical activity of a patient, the electrode configured to produce signals representative of sensed electrical activity of said patient, the electrode sensor portion having a first contact surface and a second contact surface opposite the first contact surface, wherein the electrode sensor portion is configured such that said electrical activity of said patient can be sensed using the at least one electrode at the first contact surface or at the second contact surface; and
a portion connector attached to NIRS sensor portion and to the electrode sensor portion, wherein the portion connector is configured so that the electrode sensor portion can be selectively disposed on the first lateral side of the NIRS sensor portion with the first contact surface disposed to engage a skin surface of the patient or on the second lateral side of the NIRS sensor portion with the second contact surface disposed to engage the skin surface of the patient; and
a base unit having a system controller in communication with the at least one physiological sensor assembly, the controller including at least one processor and a memory device configured to store instructions, which instructions when executed cause the controller to:
control the NIRS sensor portion of the at least one physiological sensor assembly NIRS sensing device to sense a patient's tissue and produce information relating to an oxygen saturation level of the sensed patient tissue; and
determine information relating to electrical activity within the sensed tissue of the patient.

18. The system of claim 17, wherein the information relating to electrical activity within the sensed tissue of the patient is electroencephalography (EEG) information.

19. The system of claim 17, wherein the at least one physiological sensor assembly includes a first sensor assembly and a second sensor assembly that are the same, and the first sensor assembly is configured in a first arrangement for application to a left side of a said patient forehead and the second sensor assembly is configured in a second arrangement for application to a right side of the patient forehead.

20. The system of claim 19, wherein in the first arrangement the first sensor assembly has the electrode sensor portion disposed on the first lateral side of the NIRS sensor portion of the first sensor assembly, and in the second arrangement the second sensor assembly has the electrode sensor portion disposed on the second lateral side of the NIRS sensor portion of the second sensor assembly.

21. The system of claim 17, wherein the at least one physiological sensor assembly includes a first sensor assembly and a second assembly that are the same, and the first sensor assembly is configured in a first arrangement with the electrode sensor portion disposed on the first lateral side of the NIRS sensor portion of the first sensor assembly, and the second sensor assembly is configured in a second arrangement with the electrode sensor portion disposed on the second lateral side of the NIRS sensor portion of the second sensor assembly.
